(19) United States
(12) Patent Application Publication
(10) Pub. No.: US 2025/0368593 A1
(43) Pub. Date: Dec. 4, 2025

(54) PROCESS FOR PURIFYING MONO-ETHYLENE GLYCOL

(71) Applicant: CARBIOS, CLERMONT-FERRAND (FR)

(72) Inventors: GUILLAUME ROLLAND, FOUR (FR); AMOKRANE BOUFARES, VILLEURBANNE (FR); ANTOINE SEVENIER, POMPIGNAC (FR)

(21) Appl. No.: 18/874,147

(22) PCT Filed: Jun. 13, 2023
(86) PCT No.: PCT/EP2023/065814

§ 371 Date: Dec. 12, 2024

(30) Foreign Application Priority Data
Jun. 13, 2022 (EP) 22305861.1

(51) Int. Cl. C07C 29/80; C07C 29/09; C08J 11/10; C08J 11/14; C08J 11/16

(52) U.S. Cl. CPC C07C 29/80; C07C 29/095; C08J 11/105; C08J 11/14; C08J 11/16; C08J 2367/06

(57) ABSTRACT

The present invention relates to a process for purifying and recovering the mono-ethylene glycol (MEG) from a solution obtained from the depolymerization of at least one polyester having at least one unit of MEG. This solution is preferably obtained from an enzymatic depolymerization under alkaline conditions of polyethylene terephthalate (PET) included in a plastic product. The invention also relates to a process for recycling a polymer-containing material, such as plastic product, comprising at least one polyester having at least one unit of MEG, such as PET, and recovering the monomers thereof.

### TECHNICAL FIELD

The present invention relates to a process for purifying and recovering the mono-ethylene glycol (MEG) from a solution obtained from the depolymerization of at least one polyester having at least one unit of MEG. Preferably, the solution is obtained from an enzymatic depolymerization under alkaline conditions of polyethylene terephthalate (PET) included in a plastic product. In addition, the invention relates to a process for recycling polymer-containing material, such as a plastic product, comprising at least one polyester having at least one unit of MEG, such as PET, and recovering the monomers thereof. The process of the invention is particularly useful for degrading a plastic product comprising polyethylene terephthalate.

### BACKGROUND

Plastics are inexpensive and durable materials, which can be used to manufacture a variety of products that find uses in a wide range of applications (food packaging, textiles, etc.). Therefore, the production of plastics has increased dramatically over the last decades. Moreover, most of them are used for single-use disposable applications, such as packaging, agricultural films, disposable consumer items or for short-lived products that are discarded within a year of manufacture. Because of the durability of the polymers involved, substantial quantities of plastics are piling up in landfill sites and in natural habitats worldwide, generating increasing environmental problems. For instance, in recent years, polyethylene terephthalate (PET), an aromatic polyester produced from terephthalic acid and mono-ethylene glycol, has been widely used in the manufacture of several products for human consumption, such as food and beverage packaging (e.g.: bottles, convenience-sized soft drinks, pouches for alimentary items) or textiles, fabrics, rugs, carpets, etc.

Different solutions, from plastic degradation to plastic recycling, have been studied to reduce environmental and economic impacts correlated to the accumulation of plastic waste. Mechanical recycling technology remains the most-used technology, but it faces several drawbacks. Indeed, it requires an extensive and costly sorting and it leads to downgrading applications, due to an overall loss of molecular weight during the process and uncontrolled presence of additives in the recycled products. The current recycling technologies are also expensive. Consequently, recycled plastic products are generally non-competitive compared to virgin plastic.

Chemical and enzymatic recycling of plastic products have also been developed and described (e.g WO 2014/079844, WO 2015/097104, WO 2015/173265, WO 2017/198786, and WO 2020/094646). These technologies allow the chemical constituents of the polymer to be recovered in the form of monomers and/or oligomers. Growing interest is focused on the optimum process development of hydrolysis of polyester waste, like PET waste, to recover terephthalic acid (TA) and mono-ethylene glycol (MEG). Several recycling methods, including chemical recycling or recycling of polyesters using hydrolyzing-enzymes, have been reported. Processes for recovering TA have been already described (e.g WO 2020/094661) and typically, the purification of the MEG is performed by distillation. The recovered monomers/oligomers may then be purified and used to re-manufacture plastic items with equivalent quality to virgin plastic items, so that such processes lead to an infinite recycling of plastics.

However, a drawback of the depolymerization of polyesters, like PET, e.g., the alkaline depolymerization of PET, is that it generates in the reaction solution high amounts of dicarboxylic acid salts, coproducts (e.g., DEG, TEG) and other inorganics impurities which are not completely removed during the TA purification process. As a result, at the end of the process the recovered mono-ethylene glycol (MEG) comprises a substantial rate of impurities, preventing its further reuse for the manufacture of qualitative and transparent plastic products.

It is an aim of the present invention to recover purified MEG from the depolymerization of polyesters or polyester-containing material. According to EP1160228, ethylene glycols, such as MEG, produced from the reaction of ethylene oxide with water are separated from an aqueous mixture with organic acids, salts and unidentified UV light absorbers. The suitability of the process disclosed in EP1160228 for recovering MEG from a depolymerization solution of polyesters, like PET, is neither disclosed nor suggested in this document. Especially, a depolymerization solution of a polyester comprising at least one unit of MEG comprises components different from those of an aqueous solution as that disclosed in EP1160228. The depolymerization solution may comprise impurities such as derivatives of the other units of the depolymerized polymer, such as terephthalic acid or salts thereof when the polyester is PET, and/or heavy impurities.

By working on this issue, the inventors surprisingly evidenced that implementation of a process comprising specific steps in a defined order allows obtaining a highly pure MEG from a solution obtained from the depolymerization of at least one polyester having at least one unit of MEG. The purity of the obtained MEG is far higher than that obtained by classical distillation purification, and may even be as high as the purity required in the specifications existing for petrochemical MEG.

### SUMMARY OF THE INVENTION

The inventors have discovered that it is possible to set up a purification process that leads to a highly purified MEG, suitable to be repolymerized, from the depolymerization of a polyester having at least one unit of MEG. More particularly, the inventors have developed a process that makes it is possible to recover highly pure MEG from the hydrolysis of a polyester having at least one unit of MEG. In addition, the inventors have discovered that this purification process can be implemented for purifying MEG from a solution obtained from chemical as well as from biological depolymerizations of at least one polyester having at least one unit of MEG.

It is the merits of the inventors to have determined a particular series of steps to obtain highly purified MEG from a solution comprising ethylene glycol and salts as depolymerization products and that leads to the removal of all undesirable impurities in the final MEG. The process of the invention allows recovering the MEG monomers that formed original polymers of a polyester, so that said monomers may be reprocessed to synthesize new polymers of the same type or of another type.

In this regard, it is an object of the invention to provide a process for purifying mono-ethylene glycol (MEG) from a depolymerization solution of at least one polyester having at least one unit of MEG, the process comprises the following steps of:
a) Submitting said depolymerization solution to at least one evapo-condensation step to obtain a bottom fraction and a condensed overhead fraction,
b) Contacting the condensed overhead fraction obtained in step (a) with a resin, to obtain a solution,
c) Submitting the solution obtained in step (b) to at least one distillation step to obtain a distillate, and
d) Recovering the purified MEG from the distillate obtained in step (c).

In an embodiment, the polyester having at least one unit of MEG is selected from the group consisting of polyethylene terephthalate (PET), polyethylene adipate (PEA), polyethylene-2,5-furanoate (PEF), and polyethylene naphthalate (PEN). In a particular embodiment, the polyester having at least one unit of MEG is PET.

In an embodiment, the depolymerization, from which the depolymerization solution submitted to the process of the invention is obtained, is either a biological or a chemical depolymerization. In a preferred embodiment, the biological or the chemical depolymerization is a hydrolysis, preferably a hydrolysis in alkaline conditions (i.e., an alkaline enzymatic depolymerization or an alkaline chemical depolymerization such as saponification), even more preferably an alkaline enzymatic depolymerization.

In a preferred embodiment, the depolymerization solution of at least one polyester having at least one unit of MEG comprises at least MEG, water and heavy impurities and may further comprises acid traces and/or salts.

In an embodiment, the depolymerization solution of at least one polyester having at least one unit of MEG is obtained from a reaction solution of a depolymerization of at least one polyester having at least one unit of MEG comprising dicarboxylic acids salts and MEG and submitted to one or more of the steps selected from the group consisting of filtration, decoloration, precipitation and evapo-concentration.

Advantageously, step (a) comprises a first evapo-condensation step, and further comprises submitting the bottom fraction obtained from the first evapo-condensation step to a second evapo-condensation step, wherein the condensed overhead fraction obtained from the second evapo-condensation step is mixed with the condensed overhead fraction obtained from the first evapo-condensation step, and wherein the mixed condensed overhead fractions are submitted to step (b).

In a preferred embodiment, the bottom fraction obtained from the first evapo-condensation step is filtrated prior to being submitted to the second evapo-condensation step.

In a preferred embodiment, step (b) is performed by contacting the condensed overhead fraction obtained in step (a) with an ion exchange resin, preferably with a strong anion exchange resin.

In a preferred embodiment, step (c) consists in two distillation steps.

In a particular embodiment, the depolymerization solution is obtained from the enzymatic depolymerization of PET wherein the pH of the reaction medium is regulated between 6.5 and 9 by addition of a base in said reaction medium. The TA salts produced from said depolymerization are removed from the solution by precipitation and filtration as described in WO 2020/094661 in order to obtain the reaction solution to be submitted to the process of the invention.

It is an object of the invention to provide a process for purifying MEG from a depolymerization solution of at least one polyester having at least one unit of MEG, wherein said process comprises or consists in the following steps of:
a. Submitting said depolymerization solution to at least one evapo-condensation step, preferably two evapo-condensation steps both performed in a thin-film evaporator, to obtain a bottom fraction and a mixture of condensed overhead fractions,
b. Contacting the condensed overhead fractions obtained in step (a) with a strong anion exchange resin to obtain a solution,
c. Submitting the solution obtained in step (b) to a double distillation, and
d. Recovering purified MEG from the distillate obtained in step (c).

It is also an object of the invention to provide a process for recycling a polymer-containing material, such as a plastic product, comprising at least one polyester having at least one unit of MEG, the process comprising the following steps of:
a.1) Submitting the polyester having at least one unit of MEG to a depolymerization step to obtain a reaction solution comprising dicarboxylic acid salts, MEG and solid components,
b.1) Submitting the reaction solution obtained in step (a.1) to a filtration to remove solid components and obtain a filtrate,
c.1) Purifying the filtrate obtained in step (b.1) to obtain a purified filtrate,
d.1) Precipitating the dicarboxylic acid by acidification of the purified filtrate obtained in step (c.1), to obtain a slurry,
e.1) Submitting the slurry obtained in step (d.1) to a filtration to remove the precipitated dicarboxylic acid, to obtain a filtrate,
f.1) Submitting the filtrate obtained in step (e.1) to at least one evapo-concentration step to obtain an MEG concentrated solution,
g.1) Purifying the MEG of the concentrated solution obtained in step (f.1) by the process according to the invention, to obtain purified MEG.

Advantageously, in step (a.1) the depolymerization is a hydrolysis in alkaline conditions, preferably an alkaline enzymatic depolymerization. Advantageously, in step (c.1) the purification of the filtrate obtained in step (b.1) is carried out through one or several steps selected from ultrafiltration, adsorption on activated carbon, submission to an ion exchange resin and chromatography.

In an embodiment, the polymer-containing material comprising at least one polyester having at least one unit of MEG is a plastic product comprising at least one polyester having at least one unit of MEG. In a particular embodiment, when the polyester having at least one unit of MEG is a PET, the dicarboxylic acid is terephthalic acid (TA) and the dicarboxylic acid salts are terephthalic acid salts.

It is also an object of the invention to use the purified MEG obtained by a process according to the invention to produce a polyester containing at least one unit of MEG.

These and the other objects and embodiments of the invention will become more apparent after the detailed description of the invention, including preferred embodiments thereof given in general terms.

### DETAILED DESCRIPTION OF THE INVENTION

The present invention refers to a process for purifying mono-ethylene glycol (MEG) from a solution obtained from the depolymerization of at least one polyester having at least one unit of MEG and allows recovering the MEG monomers that formed said original polyester, so that said monomers may be reprocessed to synthesize new polyesters.

The present invention particularly allows to remove part or all of, preferably all of, the remaining dicarboxylic acid salts (such as TA salts if polyester is PET) and other inorganic impurities, as well as coproducts such as di-ethylene glycol (DEG) and tri-ethylene glycol (TEG) present in the solution obtained from the depolymerization process.

The present invention refers also to a recycling process for recycling a polymer-containing material, such as a plastic product, comprising at least one polyester having at least one unit of MEG, to generate and recover TA salts along with a purified MEG monomer.

### Definitions

In the context of the invention, a “polymer-containing material” or “polymer-containing product” refers to a product, such as a plastic product, comprising at least one polymer in crystalline, semi-crystalline or totally amorphous form. In a particular embodiment, the polymer-containing material refers to any item made from at least one plastic material, such as plastic sheet, tube, rod, profile, shape, film, massive block, fiber, etc., which contains at least one polyester having at least one unit of MEG, and possibly other substances or additives, such as plasticizers, mineral or organic fillers. In another particular embodiment, the polymer-containing material refers to a plastic compound, or plastic formulation, in a molten or solid state, suitable for making a plastic product. In another particular embodiment, the polymer-containing material refers to textile, fabrics or fibers comprising at least one polymer. In another particular embodiment, the polymer-containing material refers to plastic waste or fiber waste comprising at least one polymer. Particularly, the polymer-containing material is a plastic product.

Within the context of the invention, the terms “plastic article” or “plastic product” are used to refer to any item or product comprising at least one polymer, such as plastic sheet, tube, rod, profile, shape, massive block, fiber, etc. Preferably, the plastic article is a manufactured product, such as rigid or flexible packaging (bottle, trays, cups, etc.), agricultural films, bags and sacks, disposable items or the like, carpet scrap, fabrics, textiles, etc. The plastic article may contain additional substances or additives, such as plasticizers, minerals, organic fillers or dyes. In the context of the invention, the plastic article may comprise a mix of semi-crystalline and/or amorphous polymers and/or additives.

A “polymer” refers to a chemical compound or mixture of compounds whose structure is constituted of multiple repeating units (i.e., “monomers”) linked by covalent chemical bonds. Within the context of the invention, the term “polymer” refers to such chemical compound used in the composition of a plastic product.

A “recycling process” in relation to a plastic product refers to a process by which at least one polyester of said plastic product is degraded to produce at least one type of monomers and/or oligomers, which are retrieved in order to be reused. Optionally, said monomers and/or oligomers are suitable for further re-polymerization. In the context of the invention, the plastic product comprises at least one polyester having at least one unit of MEG is subjected to depolymerization and yields to at least MEG monomers.

The term “polyester” refers to a polymer that contains the ester functional group in its main chain. Ester functional group is characterized by a carbon bound to three other atoms: a single bond to a carbon, a double bond to an oxygen, and a single bond to an oxygen. The singly bound oxygen is bound to another carbon. According to the composition of their main chain, polyesters can be aliphatic, aromatic or semi-aromatic. Polyester can be homopolymer or copolymer.

The term “polyester having at least one unit of MEG” refers to a polyester formed from MEG and dicarboxylic acid monomers. Examples of polyesters having at least one unit of MEG comprise polyethylene terephthalate (PET), polyethylene adipate, polyethylene-2,5-furanoate and polyethylene naphthalate. Polyethylene terephthalate is a semi-aromatic copolymer composed of two monomers: terephthalic acid and ethylene glycol. Polyethylene adipate is an aliphatic copolymer composed of adipic acid and ethylene glycol monomers. Polyethylene-2,5-furanoate is a semi-aromatic copolymer composed of 2,5-furandicarboxylic acid and ethylene glycol monomers. Polyethylene naphthalate is a semi-aromatic copolymer composed of naphtalene-2,6-dicarboxylic acid and ethylene glycol monomers.

As used in the present application, the terms “solubilized” or “in solubilized form” designate a compound dissolved in a liquid, unlike undissolved solid forms.

The term “depolymerization” in relation to the solution obtained from the depolymerization of at least one polyester having at least one unit of MEG refers to a process by which the polyester having at least one unit of MEG has been depolymerized and/or degraded into smaller molecules, such as monomers and/or oligomers and/or any degradation products. Depolymerization processes include chemical and biological depolymerization processes.

The expressions “solution obtained from the depolymerization of at least one polyester having at least one unit of MEG” or “depolymerization solution of at least one polyester having at least one unit of MEG” or “solution of the depolymerization of at least one polyester having at least one unit of MEG”, or even “depolymerization solution” refer to the solution resulting from or obtained either directly at the end of a depolymerization step of said polyester, or after one or several treatment steps of a solution resulting from or obtained directly at the end of a depolymerization step of said polyester. A depolymerization solution according to the invention comprises components and/or impurities specific to the depolymerization process, and is thus different from any solution comprising MEG and obtained by processes different from depolymerization. Said solution comprises MEG and other degradation products. Among degradation products may be cited for instance other monomers, oligomers, and/or salts thereof. The treatment steps may be implemented to remove part of the degradation products.

According to the invention, “oligomers” refer to molecules containing from 2 to about 20 monomer units. As an example, oligomers that may be retrieved from PET include mono-2-hydroxyethyl terephthalate (MHET), bis(2-hydroxyethyl) terephthalate (BHET), 1-(2-hydroxyethyl) 4-methyl terephthalate (HEMT), dimethyl terephthalate (DMT), di-ethylene-glycol (DEG) and/or tri-ethylene-glycol (TEG).

The term “salt”, when referring to an acid salt, refers to any compound formed when the hydrogen ions of an acid are partly or completely replaced by a positive ion such as sodium, potassium, ammonium or a metal ion.

The term “dicarboxylic acids” refers to compounds containing two carboxyl functional groups-COOH. It is represented by the formula HO₂C—R—CO₂H, where R can be aliphatic and/or aromatic, preferably aromatic. As an example, terephthalic acid, isophthalic acid, adipic acid, 2,5-furandicarboxylic acid and naphthalene 2,6-dicarboxylic acid are dicarboxylic acids. Dicarboxylic acids typically originate from the units other than MEG of the polyester. Dicarboxylic acids preferably have a molecular weight of 100 g/mol or more. In an embodiment, dicarboxylic acids are different from oxalic acid.

The “dicarboxylic acid salts” are formed when replaceable hydrogen ions in dicarboxylic acids are partly or completely replaced by a positive ion such as sodium, potassium, ammonium or a metal ion. “TA salts” or “terephthalic acid salts” are included in this definition. In the context of the invention, the TA salts can comprise the disodium terephthalate C₈H₄Na₂O₄, dipotassium terephthalate C₈H₄K₂O₄, diammonium terephthalate C₈H₁₂N₂O₄, monosodium terephthalate C₈H₅NaO₄, monopotassium terephthalate C₈H₅KO₄ and/or monoammonium terephthalate C₈H₁₀NO₄.

The term “mono-ethylene glycol” or “MEG” refers to molecules represented by the formula C₂H₆O₂ (HO—CH₂—CH₂—OH) that are recovered at the end of the purification process according to the invention.

The term “purifying process” when referring to a compound refers to a process wherein the purity degree of said compound is increased. For instance, the purity of the compound before the purifying process may be lower than 70%, lower than 50%, and even lower than 30%, in weight relative to the total weight of the sample comprising the compound to be purified. For instance, the purity of the compound after the purifying process may be higher than 80%, preferably higher than 90%, more preferably higher than 95%, even more preferably higher than 99% in weight relative to the total weight of the sample comprising the purified compound.

The “heating temperature” as used in the present invention corresponds to the temperature of the heating medium which is used to convey heat from a heat source, for instance steam, either directly or through a suitable heating device, to the process medium which in turn is used in the process. The term “process temperature” or “bottom temperature” corresponds to the temperature of the bottom fraction inside the process medium (i.e., the solution obtained from the depolymerization of at least one polyester having at least one unit of MEG or the solution to be distillated). Due to the heat transfer, the heating temperature of the heating medium is generally higher than the process temperature inside the process medium. Moreover, when measuring the temperature, the margin error can range by +/−5° C., +/−4° C., +/−3° C., +/−2° C., or +/−1° C.

The “ambient temperature” or “room temperature” means a temperature between 10° C. and 30° C., particularly between 20° C. and 25° C.

The expression “comprised between X and Y” includes boundaries, unless explicitly stated otherwise. This expression means that the target range includes the X and Y values, and all values from X to Y.

### Process for Purification of the MEG According to the Invention

It is an object of the invention to provide a process for purifying mono ethylene glycol (MEG) from a depolymerization solution of at least one polyester having at least one unit of MEG, the process comprises the following steps of:
a) Submitting said depolymerization solution to at least one evapo-condensation step to obtain a bottom fraction and a condensed overhead fraction,
b) Contacting the condensed overhead fraction obtained in step (a) with a resin, to obtain a solution, and
c) Submitting the solution obtained in step (b) to at least one distillation step to obtain a distillate, and
d) Recovering the purified MEG from the distillate obtained in step (c).

This process can provide highly purified MEG from a solution obtained from the depolymerization of at least one polyester having at least one unit of MEG.

Indeed, the inventors have surprisingly discovered that the purity of the MEG recovered from a solution obtained from the depolymerization of a polyester, thanks to the process of the present invention, has been greatly improved to come close to that of the petrochemical sourced MEG. More particularly, this purification process allows to re-use of the recovered monomers of MEG suitable to be repolymerized in polyester chains.

In addition, the obtained MEG presents a low color value according to the APHA-scale, especially a low APHA index and/or a low APHA-boiling value. The APHA-boiling value may be obtained, as the APHA-index, according to ASTM-5386, after heating the sample at 198° C. for 4 hours. The APHA index of the obtained MEG is preferably lower than 5, and/or the APHA boiling value is preferably lower than 200, more preferably lower than 160, even more preferably lower than 50, in particular lower than 20.

### Step (a) of Evapo-Condensation

The process for purifying MEG comprises a step (a) wherein the solution obtained from the depolymerization of at least one polyester having at least one unit of MEG is submitted to at least one evapo-condensation step to obtain a bottom fraction and a condensed overhead fraction. According to the invention, an evapo-condensation step means an evaporation step followed by a condensation step. In an embodiment, the evapo-condensation of step (a) consists in one evaporation step and one condensing step.

The condensed overhead fraction corresponds to the fraction that evaporates during the evaporation step, which is recovered in the top part of the evaporator and condensed during the condensation step. The condensed overhead fraction classically includes the MEG, as well as other volatile molecules (such as water, DEG and TEG).

The bottom fraction corresponds to the fraction that remains at the bottom of the evaporator during the evapo-condensation step.

Advantageously, said step allows to remove salts from said condensed overhead fraction. More particularly, said step allows to recover heavy impurities at the bottom of the evaporator, mostly salts and solid impurities. In an embodiment, when the polyester submitted to the depolymerization is PET, the depolymerization process is an alkaline hydrolysis, preferably an enzymatic or a chemical alkaline hydrolysis wherein NaOH is used. In such case, said step allows to recover mostly TA salts and other salts such as Na₂SO₄ at the bottom of the evaporator.

The evaporation step allows to separate out the one or more dissolved salts contained in the solution obtained from the depolymerization of at least one polyester having at least one unit of MEG. This step is carried out by heating said solution under vacuum until the desired evaporation rate is reached. The evaporation rate is the ratio between the amount evaporated and the amount supplied. A person skilled in the art knows how to adapt the desired evaporation rate so as not to clog the bottom of the evaporator with the salts which precipitate. The salts remain in the bottom fraction and the MEG is vaporized, as well as other volatile molecules (such as water, DEG and TEG). The vapor enters the condensation step to lead to an overhead fraction.

The separated salts can be, for example, selected from the group consisting of dicarboxylic acids salts, such as terephthalic acid salts and salts of other degradation products of the polyester. Among the degradation products of the polyester, which salts may be separated at the evapo-condensation step (a), may be cited for instance methyl-2-hydroxyethyl terephthalate (MHET), bis(2-hydroxyethyl) terephthalate (BHET) and isophthalic acid (IPA) salts.

The evaporation of the evapo-concentration of step (a) can be performed in any suitable evaporator, such as a thin-film evaporator, a batch evaporator, a forced circulation evaporator or a flash evaporator, preferably in a thin-film evaporator. Subsequently, the condensation of the vaporized overhead fraction can be performed by passing the overhead fraction through a heat-exchanger, preferably a tubular heat-exchanger or a plate heat-exchanger with a cooling medium at a temperature below 50° C. Preferably, the heat-exchanger is a condensing unit, more preferably a water-cooled condensing unit.

Step (a) may comprise, or consist of, one or several evapo-condensation steps depending on the content of the solution submitted to step (a), its volume and/or the type of evaporator used.

One skilled in the art is able to adjust the evaporation conditions, such as the temperature, the pressure, and/or the evaporation rate, depending among others on the sample to be evaporated and the used evaporator. For instance, if the evaporation is performed in a thin-film evaporator, the evaporation rate is preferably limited to 90% by controlling the heating or the process temperature during the evaporation to avoid the formation of dry zones on the exchanger surface.

According to the invention, the evapo-condensation step is operated through controlling the process pressure (inside the process medium) simultaneously with either the heating temperature or the process temperature.

When the temperature of the heating medium is controlled, the heating temperature during the evaporation is below 200° C. below 195° C. below 193° C. below 190° C. below 185° C. below 180° C. below 170° C. preferably below 195° C. and the pressure is of 15 mbar abs or more, such as 20 mbar abs or more, such as 30 mbar abs or more, such as 40 mbar abs or more, such as 50 mbar abs or more, such as 60 mbar abs or more, such as 70 mbar abs or more, such as 80 mbar abs or more, such as 85 mbar abs or more, such as 90 mbar abs or more, such as 95 mbar abs or more, such as 98 mbar abs or more, such as 99 mbar abs or more, such as 100 mbar abs or more. The pressure may be of 1000 mbar abs or less, such as 500 mbar abs or less, such as 300 mbar abs or less, such as 200 mbar abs or less. In some embodiments, the heating temperature during the evaporation is below 200° C. below 195° C. below 193° C. below 190° C. below 185° C. below 180° C. below 170° C. preferably below 195° C. and the pressure is comprised between 50 mbar abs and 1 000 mbar abs, preferably comprised between 50 mbar abs and 500 mbar abs, more preferably between 50 and 300 mbar abs, even more preferably between 50 and 200 mbar abs. In some embodiments, the evaporation of step (a) is operated at a heating temperature comprised between 80° C. and 200° C. preferably between 100° C. and 195° C. more preferably between 120° C. and 193° C. even more preferably between 140° C. and 193° C. and the pressure is of 15 mbar abs or more, such as 20 mbar abs or more, such as 30 mbar abs or more, such as 40 mbar abs or more, such as 50 mbar abs or more, such as 60 mbar abs or more, such as 70 mbar abs or more, such as 80 mbar abs or more, such as 85 mbar abs or more, such as 90 mbar abs or more, such as 95 mbar abs or more, such as 98 mbar abs or more, such as 99 mbar abs or more, such as 100 mbar abs or more. The pressure may be of 1000 mbar abs or less, such as 500 mbar abs or less, such as 300 mbar abs or less, such as 200 mbar abs or less. In some embodiments, the evaporation of step (a) is operated at a heating temperature comprised between 80° C. and 200° C. preferably between 100° C. and 195° C. more preferably between 120° C. and 193° C. even more preferably between 140° C. and 193° C. and the pressure is comprised between 50 mbar abs and 1 000 mbar abs, preferably comprised between 50 mbar abs and 500 mbar abs, more preferably between 5 and 300 mbar abs, even more preferably between 50 and 200 mbar abs.

Alternatively, when the temperature inside the process medium is controlled, the process temperature during the evaporation is below 195° C. below 190° C. below 180° C. preferably below 170° C. more preferably comprised between 80° C. and 200° C. more preferably between 100° C. and 175° C. and the pressure is of 15 mbar abs or more, such as 20 mbar abs or more, such as 30 mbar abs or more, such as 40 mbar abs or more, such as 50 mbar abs or more, such as 60 mbar abs or more, such as 70 mbar abs or more, such as 80 mbar abs or more, such as 85 mbar abs or more, such as 90 mbar abs or more, such as 95 mbar abs or more, such as 98 mbar abs or more, such as 99 mbar abs or more, such as 100 mbar abs or more. The pressure may be of 1000 mbar abs or less, such as 500 mbar abs or less, such as 300 mbar abs or less, such as 200 mbar abs or less, such as 100 mbar abs or less. In an embodiment, the process temperature during the evaporation is below 195° C., below 190° C., below 180° C., preferably below 170° C., more preferably comprised between 80° C. and 200° C., more preferably between 100° C. and 175° C. and the pressure is comprised between 50 mbar abs and 1 000 mbar abs, preferably comprised between 50 mbar abs and 500 mbar abs, more preferably between 50 and 300 mbar abs, even more preferably between 50 and 200 mbar abs.

In an embodiment, the evaporation of the first and/or of the second evapo-condensation steps of step (a) is/are performed under vacuum conditions, preferably at a pressure of 15 mbar abs or more, preferably comprised between 15 and 500 mbar abs, such as between 50 and 500 mbar abs, more preferably at 100 mbar abs.

In some embodiments, the step (a) comprises 1, 2, 3, 4, or 5 evapo-condensation steps. Preferably, the step (a) comprises 1, 2, 3 or 4 evapo-condensation steps, more preferably two evapo-condensation steps.

In some embodiments, the step (a) comprises or consists in one evapo-condensation step. In some embodiments, the step (a) comprises or consists in one evapo-condensation step which is performed in a forced circulation evaporator.

When the step (a) comprises more than one evapo-condensation steps, each evapo-condensation step is performed on the bottom fraction of the previous evapo-condensation step. The condensed overhead fractions obtained after each condensation step are preferably mixed prior to being submitted to step (b).

When the step (a) comprises more than one evapo-condensation steps, the last evapo-condensation step is, preferably, performed in a thin-film evaporator. In some embodiments, the first evapo-condensation step is performed in a forced circulation evaporator and the second one is performed in a thin-film evaporator. In some embodiments, the first and/or the second evapo-condensation step is/are performed in a batch evaporator.

In another embodiment, further to the first evapo-condensation step in step (a), step (a) comprises one additional evapo-condensation step, wherein the bottom fraction obtained from the first evapo-condensation step is submitted to a second evapo-condensation step, and wherein the condensed overhead fractions obtained from the first and second evapo-condensation steps are mixed prior to being submitted to step (b). This additional evapo-condensation step increases the yield of the recovered MEG.

In a particular embodiment, the bottom fraction obtained from the first evapo-condensation step is filtrated prior to being submitted to the second evapo-condensation step. Particularly, the evaporation of the second evapo-condensation step is performed in a thin-film evaporator.

In an embodiment, the first and the second evapo-condensation steps are performed in a thin-film evaporator. Particularly, the temperature of the additional evaporation is higher than the temperature of the first evaporation, the two evaporations being conducted at the same pressure.

In a particular embodiment, step (a) consists of two evapo-condensation steps, wherein the bottom fraction obtained from the first evapo-condensation step is submitted to a second evapo-condensation step, and wherein the condensed overhead fractions obtained from the first and second evapo-condensation steps are mixed prior to being submitted to step (b).

Particularly, the evaporation of the first evapo-condensation step is conducted at a process temperature between 90° C. and 180° C., between 100° C. and 170° C., between 120° C. and 170° C., between 90° C. and 150° C., between 100° C. and 155° C., between 110° C. and 140° C., between 110° C. and 125° C. preferably between 115° C. and 120° C., at a pressure of 15 mbar abs or more, such as comprised between 15 and 500 mbar abs, such as comprised between 50 and 500 mbar abs, between 50 and 300 mbar abs, preferably at 100 mbar abs, and the evaporation of the second evapo-condensation step is conducted at a process temperature between 120° C. and 150° C., between 120° C. and 155° C., between 130° C. and 155° C., between 130° C. and 150° C., between 135° C. and 150° C., between 135° C. and 145° C., at a pressure of 15 mbar abs or more, such as comprised between 15 and 500 mbar abs, such as comprised between 50 and 500 mbar abs, preferably at 100 mbar abs.

In a particular embodiment, the step (a) consists of two evapo-condensation steps wherein:
(1) The evaporation of the first evapo-condensation step is operated at a process temperature comprised between 100° C. and 180° C., such as between 110° C. and 135° C., preferably between 110° C. and 125° C., more preferably 118° C., at a pressure of 15 mbar abs or more, such as comprised between 15 and 500 mbar abs, preferably comprised between 50 and 500 mbar abs, more preferably comprised between 50 and 120 mbar abs, such as between 80 and 120 mbar abs, and
(2) The evaporation of the second evapo-condensation is operated at a process temperature comprised between 130° C. and 150° C., preferably 144° C., at a pressure of 15 mbar abs or more, such as comprised between 15 and 500 mbar abs, preferably between 50 and 500 mbar abs, more preferably comprised between 50 and 120 mbar abs, such as between 80 and 120 mbar abs.

In a preferred embodiment, an evaporation step is performed at 100 mbar abs. In a particular embodiment, both evaporations are performed at the same pressure, preferably at 100 mbar abs.

Particularly, the evaporation of the first evapo-condensation step is conducted at a heating temperature between 150° C. and 180° C., between 155° C. and 170° C., between 160° C. and 170° C., at a pressure of 5 mbar abs or more, such as comprised between 5 and 500 mbar abs, such as comprised between 50 and 500 mbar abs, between 50 and 300 mbar abs, preferably at 100 mbar abs; and the evaporation of the second evapo-condensation step is conducted at a heating temperature between 150° C. and 200° C., between 155° C. and 195° C. preferably between 165° C. and 195° C., at a pressure of 5 mbar abs or more, such as comprised between 5 and 500 mbar abs, such as comprised between 50 and 500 mbar abs, preferably at 100 mbar abs. Preferably, the two evaporation steps are conducted at the same at 100 mbar abs.

In a particular embodiment, the step (a) consists of two evapo-condensation steps wherein:
(1) The evaporation of the first evapo-condensation step is operated at a heating temperature comprised between 155 and 170° C., preferably between 160° C. and 170° C., at a pressure of 5 mbar abs or more, such as comprised between 5 and 500 mbar abs, such as comprised between 50 and 500 mbar abs, preferably comprised between 50 and 120 mbar abs, such as between 80 and 120 mbar abs, and
(2) The evaporation the second evapo-condensation is operated at a heating temperature comprised between 155° C. and 195° C., preferably between 165° C. and 195° C., at a pressure of 5 mbar abs or more, such as comprised between 5 and 500 mbar abs, such as comprised between 50 and 500 mbar abs, preferably comprised between 50 and 120 mbar abs, such as between 80 and 120 mbar abs.

Advantageously, the condensed overhead fraction(s) recovered from step (a) comprise(s) at least 60% of MEG, preferably at least 70% of MEG, more preferably at least 80% of MEG.

Step (b) of Contacting the Condensed Overhead Fraction(s) Obtained in Step (a) with a Resin

In this step, the condensed overhead fraction or the mixed condensed overhead fractions of the previous step (a) is contacted with, preferably passed through, a resin, to obtain a solution.

The inventors evidenced that contacting the overhead fraction(s) obtained at step (a) with a resin afforded a solution comprising MEG with a lower acidity, even when the solution submitted to step (a) comprises acidic compounds and/or traces. In a preferred embodiment, the condensed overhead fraction(s) of step (a) is contacted with, preferably passed through, an ion exchange resin, preferably a strong anion exchange resin. One skilled in the art is able to select the suitable ion exchange resin to be implemented and to adapt the flow rate depending on the content of the overhead fraction(s) obtained at step (a). Among the anion exchange resins may be cited the anion exchange resins sold under the tradename Purolite R comprising quaternary ammonium groups, such as resins Purolite® A500MBPlusOH or Purolite® A860. Preferably, the ion exchange resin is the resin Purolite R A860. The anion exchange resin may be for instance a polyacrylic crosslinked with divinylbenzene anion exchange resin, or a polystyrene crosslinked with divinylbenzene anion exchange resin. The use of an anion exchange resin allows a higher purity and improves the UV transmissions of the purified MEG.

According to the invention, this step is preferably conducted in a continuous mode at room temperature.

### Step (c) of Distillation

According to the invention, the solution obtained in step (b) is submitted to at least one distillation step (c) to obtain a distillate. Step (c) aims at separating the monomers of MEG from the oligomers such as DEG and TEG that are present in the solution. Furthermore, this step allows to lower the water content of the solution.

As for the evapo-condensation step, this step is operated through controlling the process pressure (inside the process medium) simultaneously with either the heating temperature or the process temperature.

Step (c) may comprise, or consist of, one or several distillation steps. In some embodiments, step (c) comprises 1, 2, 3, or 4 distillation steps.

The skilled person will know how to adapt the temperature and/or pressure to perform the distillations. Especially, depending on the number of distillation steps to be implemented in step (c), one skilled in the art will know how to adapt the temperature and the pressure of each distillation step in order for the distillate to contain MEG.

When the temperature of the heating medium is controlled, the heating temperature during the distillation step(s) is/are below 250° C., below 220° C., below 200° C., preferably below 190° C. at a pressure of 15 mbar abs or more, such as 20 mbar abs or more, such as 22 mbar abs or more, such as 25 mbar abs or more, such as 27 mbar abs or more, such as 40 mbar abs or more, such as 50 mbar abs or more, such as 80 mbar abs or more, such as 90 mbar abs or more, such as 100 mbar abs or more, such as 120 mbar abs or more, such as 150 mbar abs or more, such as 170 mbar abs or more, such as 180 mbar abs or more, such as 195 mbar abs or more, such as 200 mbar abs or more, such as 205 mbar abs. The pressure may be of 1000 mbar abs or less, such as 500 mbar abs or less, such as 300 mbar abs or less, such as 220 mbar abs or less, such as 210 mbar abs or less, such as 205 mbar abs or less, such as 200 mbar abs or less. Preferably, the pressure is between 15 and 300 mbar abs, such as between 20 and 250 mbar abs. In some embodiments, the heating temperature during the distillation step(s) is/are below 250° C., below 220° C., below 200° C., preferably below 190° C. and the pressure is comprised between 50 and 300 mbar abs, preferably between 100 and 250 mbar abs. In some embodiments, the distillation step(s) is/are operated at a heating temperature comprised between 140° C. and 250° C., between 150° C. and 250° C., between 155° C. and 220° C., between 160° C. and 250° C., between 170° C. and 250° C., between 160° C. and 220° C. preferably between 170° C. and 210° C.

Alternatively, when the temperature inside the process medium is controlled, the process temperature (bottom temperature) is comprised between 110° C. and 200° C., 125° C. and 180° C., between 130° C. and 180° C., between 135° C. and 175° C. and at a pressure of 15 mbar abs or more, such as 20 mbar abs or more, such as 22 mbar abs or more, such as 25 mbar abs or more, such as 27 mbar abs or more, such as 40 mbar abs or more, such as 50 mbar abs or more, such as 80 mbar abs or more, such as 90 mbar abs or more, such as 100 mbar abs or more, such as 120 mbar abs or more, such as 150 mbar abs or more, such as 170 mbar abs or more, such as 180 mbar abs or more, such as 195 mbar abs or more, such as 200 mbar abs or more, such as 205 mbar abs. The pressure may be of 1000 mbar abs or less, such as 500 mbar abs or less, such as 300 mbar abs or less, such as 220 mbar abs or less, such as 210 mbar abs or less, such as 205 mbar abs or less, such as 200 mbar abs or less. Preferably, the pressure is between 15 and 300 mbar abs, such as between 20 and 250 mbar abs. Preferably, the pressure is between 15 and 300 mbar abs. In some embodiments, the process temperature (bottom temperature) is comprised between 110° C. and 200° C., 125° C. and 180° C., between 130° C. and 180° C., between 135° C. and 175° C. and the pressure is comprised between 50 and 300 mbar abs, preferably between 100 and 250 mbar abs.

In an embodiment, step (c) consists in one distillation step. In a particular embodiment, step (c) consists in one distillation step wherein the MEG is directly recovered via a side take-off. Accordingly, when the temperature inside the process medium is controlled, the distillation is implemented at a process temperature (bottom temperature) comprised between 120° C. and 190° C., preferably comprised between 130° C. and 180° C., more preferably between 140° C. and 180° C. and at a pressure between 15 and 300 mbar abs, such as between 50 and 300 mbar abs, preferably between 90 and 210 mbar abs, more preferably at 100 mbar abs. One skilled in the art knows how to adapt the side take-off to recover the MEG.

In another embodiment, the solution obtained in step (b) is submitted to at least two respective distillation steps. In a particular embodiment, step (c) consists in two distillation steps, preferably, wherein the pressure of the second distillation is lower than the pressure of the first distillation.

For instance, when two distillation steps are implemented:
(1) the first distillation is performed at a temperature (bottom temperature) comprised between 120° C. and 170° C., preferably between 130° C. and 160° C. and at a pressure between 100 and 300 mbar abs, preferably between 140 and 230 mbar abs; and
(2) the second distillation is performed at a temperature (bottom temperature) comprised between 130° C. and 220° C., preferably between 130° C. and 180° C., and at a pressure between 50 and 300 mbar abs, preferably between 90 and 210 mbar abs.

According to this particular embodiment of the invention:
the first distillation allows to remove the water and light components in the overhead fraction while the MEG, DEG and TEG remain in the bottom fraction, and
the second distillation, performed on the bottom fraction obtained from the first distillation, allows to separate MEG in the overhead fraction (distillate) while the heavier components like DEG and TEG remain in the bottom fraction. In such classical distillation, the distillate containing MEG is recovered at the top of the column.

In a preferred embodiment, step (c) comprises or consists in two respective distillation steps, wherein the first distillation is performed in the same conditions as indicated above, and wherein the second distillation is a distillation with two sections, wherein the MEG is recovered by a side take-off at the upper part of the column. By “distillation with two sections”, it is meant a specific arrangement of the distillation column by addition of a supplementary upper section on the second distillation column, wherein the distillate containing MEG is not recovered at the very top of the column but at the upper part of the column. In this case, the eventual light species, if present, are preferably purged at the very top of the column.

In such an embodiment, the first distillation allows to remove the water and light components in the overhead fraction while the MEG, DEG and TEG remain in the bottom fraction, and the second distillation allows the DEG, TEG and heavy components to remain at the bottom, the MEG to be extracted and recovered by a side take-off localized in the first upper part of the column, and the eventual light species, if present, to be purged at the very top of the column.

In general, when the second distillation is a distillation with two sections, it is performed at a process temperature comprised between 130° C. and 200° C., preferably between 150° C. and 180° C., and at a pressure between 15 and 100 mbar abs, preferably between 20 and 90 mbar abs.

As an example:
(1) the first distillation is performed at a process temperature comprised between 120° C. and 195° C., preferably between 150° C. and 185° C., and at a pressure between 15 and 300 mbar abs, preferably between 100 and 230 mbar abs; and
(2) the second distillation, which is a distillation with two sections, is performed at a process temperature comprised between 130° C. and 200° C., preferably between 150° C. and 180° C., and at a pressure between 15 and 100 mbar abs, preferably between 20 and 90 mbar abs.

In another embodiment, step (c) comprises or consists in three respective distillation steps, wherein the first and the third distillations are performed in the same conditions as indicated above, and wherein the second distillation is a distillation with two sections as defined above.

In an embodiment, the distillation step(s) of step (c) is/are performed under vacuum conditions, preferably at a pressure between 50 and 500 mbar abs.

The distillate comprising MEG may be recovered by any suitable means from the distillation column used for implementing step (c). For instance, said distillation may comprise a side draw (side take-off) suitable for recovering MEG.

In a preferred embodiment, the process of purification is performed in order from a) to d) and the MEG is recovered after the distillations.

Step (c′) Further Steps

In an embodiment, the process of the invention further comprises a step (c′) wherein the distillate obtained in step c) is submitted to one or more steps selected from the group consisting of distillation, hydrogenation, dehydration and decoloration, preferably a distillation step and/or a decoloration step, before recovery step (d), wherein said decoloration step is preferably performed by activated carbon adsorption.

This step (c′) is optional and can be performed on the distillate obtained at step (c) before implementing recovering step (d). It allows to improve the UV transmissions of the purified MEG.

According to the invention, the distillate obtained at step c) may further be submitted to one or more of the step(s) selected from the group consisting of distillation, hydrogenation, dehydration or decoloration, preferably a step of distillation and/or a decoloration step.

In an embodiment, the distillate obtained at step c) may further be submitted to a decoloration step, wherein the distillate passes through an adsorbent. Said adsorbent may be any adsorbent known by one skilled in the art, such as activated carbon, macroporous resin, preferably activated carbon. Particularly, said step is preferably performed at room temperature. Preferably, the process further comprises a step wherein the decoloration step is performed by activated carbon adsorption.

In another embodiment, the distillate obtained at step c) may further be submitted to a step of distillation. Said distillation is performed in the same conditions (heating temperature or process temperature-pressure) as those indicated for the distillation of step (c).

In a particular embodiment, the distillate obtained at step c) may further be submitted to a distillation followed by a decoloration step.

According to the invention, it is an object of the invention to provide a process for purifying MEG from a solution obtained from the depolymerization of at least one unit of MEG, wherein said process comprises or consists in the following steps of:
a. Submitting said solution to two evapo-condensation steps, preferably wherein the second evapo-condensation step is performed in a thin-film evaporator, more preferably wherein the two evapo-condensation steps are performed in a thin-film evaporator, to obtain a bottom fraction and a mixture of condensed overhead fractions,
b. Contacting the condensed overhead fractions obtained in step (a) with an anion exchange resin to obtain a solution,
c. Submitting the solution obtained in step (b) to a double distillation to obtain a distillate, preferably wherein the second distillation is a distillation with two sections, and
d. Recovering purified MEG from the distillate obtained in step (c).

According to the invention, it is also an object of the invention to provide a process for purifying MEG from a solution obtained from the depolymerization of at least one unit of MEG, wherein said process comprises or consists in the following steps of:
a. Submitting said solution to two evapo-condensation steps, preferably wherein the second evapo-condensation step is performed in a thin-film evaporator, more preferably wherein the two evapo-condensation steps are performed in a thin-film evaporator, to obtain a bottom fraction and a mixture of condensed overhead fractions,
b. Contacting the condensed overhead fractions obtained in step (a) with an anion exchange resin to obtain a solution,
c. Submitting the solution obtained in step (b) to a double distillation to obtain a distillate, preferably wherein the second distillation is a distillation with two sections,
c′. Optionally, submitting the distillate obtained in step (c) to one or more of the further step(s) selected from the group consisting of distillation, hydrogenation, dehydration or decoloration, preferably a step of distillation and/or a decoloration step, to obtain a solution, and
d. Recovering purified MEG from the solution obtained in step (c′) or in step (c).

When the second distillation is a distillation with two sections, the step c′) of further step(s) is preferably not present. The step of distillation with two sections helps obtaining the same MEG purity without the need of the further step(s) of step c′).

According to the invention, the process can be carried out in discontinuous mode, i.e., under batch conditions or in a continuous mode, continuously from step a) to c), more preferably step a) to d). The continuous mode is preferred.

### Step (d) of Recovering the MEG

According to the invention, recovering the MEG means recovering the distillate obtained in step (c) or the solution obtained in step (c′) that contains the purified MEG. In other words, the purified MEG is comprised in the distillate, or the solution obtained at the end of step (c) or (c′) in a liquid form. Alternatively, the recovering of the MEG is performed continuously during the distillation step of step (c) or (c′) by using a side take off.

The process of MEG purification according to the invention allows to recover a highly purified MEG. In an embodiment, the obtained MEG has a purity of at least 95%, preferably at least 99%, more preferably at least 99.9%.

Solution Obtained from the Depolymerization or Depolymerization Solution

According to the invention, the solution obtained from the depolymerization of at least one polyester comprising at least one unit of MEG refers to the solution obtained or resulting either from the chemical or the biological depolymerization, preferably via hydrolysis, of said at least one polyester comprising at least one unit of MEG.

Composition of the Solution Obtained from the Depolymerization (Depolymerization Solution)

This solution may comprise the depolymerized and/or degraded molecules, such as monomers and/or oligomers and/or any degradation products and/or heavy impurities and/or acid traces and/or salts and/or water. More particularly, this solution may comprise monomers of MEG and/or dicarboxylic acids salts such as terephthalic acid salts, isophthalic acid (IPA) salts and/or salts and/or oligomers such as DEG and TEG and/or derivatives thereof including mono-2-hydroxyethyl terephthalate (MHET) and/or bis(2-hydroxyethyl) terephthalate (BHET), dimethyl terephthalate (DMT) and/or heavy impurities and/or acid traces and/or water. The content of the depolymerization solution depends on the depolymerizing processes, e.g. hydrolysis, glycolysis, methanolysis, etc.

In a preferred embodiment, the solution is a homogeneous solution exempt of any residual solids.

The term “heavy impurities” refers to mainly dissolved non-volatile impurities such as heavy metals, antimony, copper, depolymerization agent such as a catalyst or an enzyme.

By “acid traces” is meant the amount of total acid titrated with an aqueous base (KOH or NaOH) in a sample of ethylene glycol. The acidity is calculated as acetic acid equivalent in mg/kg. The test method is described in ASTM E 2679 which is particularly useful for determining low levels of acidity. In the context of this invention, the concentration of the acid traces is equivalent to less than 2 g KOH/kg, preferably less than 1100 mg KOH/kg, more preferably to a concentration of less than 700 mg KOH/kg measured according to ASTM E 2679.

In an embodiment, the solution obtained from the depolymerization of at least one polyester comprising at least one unit of MEG comprises MEG, at least one monomer chosen from the group consisting of dicarboxylic acids such as terephthalic acid (TPA) or isophthalic acid (IPA), at least one salt of such monomer, and/or salts such as Na₂SO₄ and/or oligomers such as mono-2-hydroxyethyl terephthalate (MHET).

In a particular embodiment, the solution obtained from the depolymerization of at least one polyester having at least one unit of MEG comprises MEG, water, heavy impurities and optionally further comprises acid traces and/or salts.

In a particular embodiment, the solution obtained from the depolymerization of at least one polyester having at least one unit of MEG comprises MEG, water and heavy impurities and salts such as sodium sulphate salts, dicarboxylic acid salts and/or acid traces.

In a particular embodiment, the solution is obtained from alkaline hydrolysis of at least one polyester comprising at least one unit of MEG comprises MEG and/or heavy impurities and/or water and/or acid traces and/or soluble salts such as such as sodium sulphate salts, dicarboxylic acid salts.

In a preferred embodiment, the solution obtained from the depolymerization of at least one polyester comprising at least one unit of MEG is the aqueous concentrated solution of MEG defined below.

In a particular embodiment, the solution obtained from the depolymerization of at least one polyester comprising at least one unit of MEG comprises at least one MEG and heavy impurities.

In an embodiment, the solution obtained from the depolymerization of PET comprises MEG, heavy impurities and terephthalic acid salts and eventually sodium sulphate salts and/or acid traces.

In a preferred embodiment, the solution obtained from the depolymerization of at least one polyester comprising at least one unit of MEG comprises at least 400 g/kg of MEG, at least 500 g/kg, at least 600 g/kg, at least 700 g/kg of MEG, or at least 800 g/kg of MEG based on the total weight of the solution.

Preferably the solution comprises at least 600 g/kg of MEG, preferably 700 g/kg of MEG.

The salts that may be present in the solution may be one or more of the group consisting of sodium, potassium, or ammonium salts or a mixture thereof. For example, the salts may be selected from the group consisting of Na₂SO₄, K₂SO₄, (NH₄)₂SO₄, NaCl, KCl, NH₄Cl, Na₂PO₄, K₂PO₄, (NH₄)₂PO₄, NaNO₃, KNO₃, NH₄NO₃ or mix thereof, and/or dicarboxylic acid salts.

In an embodiment, the dicarboxylic acid salts are selected from the group consisting of terephthalic salts, adipic acid salts, 2,5-furandicarboxylic acid salts and naphthalene-2,6-dicarboxylic acid salts. Preferably the dicarboxylic acid salts are TA salts.

In an embodiment, the solution comprises at least 5 g/kg of salts, preferably at least 10 g/kg, more preferably 20 g/kg of salts, based on the total weight of the aqueous solution. In an embodiment, the initial solution comprises at most 80 g/kg of salts, preferably at most 50 g/kg, more preferably at most 30 g/kg of salts, based on the total weight of the aqueous solution. Particularly, the initial solution comprises between 5 g/kg and 80 g/kg of salts, between 10 g/kg and 80 g/kg of salts, between 10 g/kg and 50 g/kg of salts.

### Chemical or Biological Depolymerization Step

The depolymerizing step, from which the solution submitted to the purifying process of the invention is obtained, targets at least one polyester having at least one unit of MEG. In an embodiment, the process of the invention is implemented with a polymer-containing material, preferably with plastic products deriving from plastic waste collection and/or post-industrial waste and comprising at least one polyester having at least one unit of MEG. More particularly, the process of the invention may be used for degrading domestic plastic wastes, including plastic bottles, plastic trays, plastic bags, plastic packaging, soft plastics and/or hard plastics, even polluted with food residues, surfactants, etc. Alternatively, or in addition, the process of the invention may be used for degrading used fibers, such as fibers coming from fabrics, textiles, tires and/or and industrial wastes. More particularly, the process of the invention may be used with PET plastic waste and/or PET fiber waste.

In a particular embodiment, the polyester having at least one unit of MEG is chosen from the group consisting of: polyethylene terephthalate (PET), polyethylene adipate (PEA), polyethylene furanoate (PEF), and polyethylene naphthalate (PEN). In a preferred embodiment, the polyester having at least one unit of MEG is PET.

Particularly, the depolymerizing step is a chemical depolymerization or a biological depolymerization. In a preferred embodiment, the depolymerization from which the solution submitted to the process of the invention is obtained, is a hydrolysis, preferably a hydrolysis in alkaline conditions, more preferably an alkaline enzymatic depolymerization or an alkaline chemical depolymerization such as saponification, even more preferably an alkaline enzymatic depolymerization.

Within the context of the invention, “hydrolysis” refers to the rupture of the ester bond by means of OH ions in the presence of water, regardless of whether the reaction is a biological or chemical depolymerization. For example, the hydrolysis of PET produces terephthalic acid (TA) and ethylene glycol (MEG). In a preferred embodiment, the hydrolysis is an alkaline hydrolysis, wherein an alkali (or a base) is employed as a reactant to break down the polyester in an aqueous media. Such alkali can be selected from NaOH, KOH, NH₄OH or LiOH. As an example, the alkaline hydrolysis of PET produces terephthalic acid (TA) salts and ethylene glycol (MEG).

In an embodiment, the depolymerizing step comprises contacting the at least one polyester having at least one unit of MEG with a depolymerizing agent. i.e., a chemical and/or a biological depolymerizing agent.

Advantageously, the depolymerization step comprising the depolymerizing agent is performed in a liquid medium as starting reaction medium, more advantageously an aqueous medium.

In an embodiment, the depolymerization of the polyester having at least one unit of MEG is a chemical depolymerization, preferably a hydrolysis. Preferably, the chemical depolymerization is an alkaline chemical hydrolysis such as a saponification.

Within the context of the invention, the term “chemical depolymerization” refers to a process by which the depolymerization of at least one polyester having at least one unit of MEG is performed by contacting the polymer with a reactant, such as methanol or water, optionally in the presence of one or more chemical agent such as a catalyst. These methods are respectively known as methanolysis and chemical hydrolysis.

Other methods include glycolysis, aminolysis and ammonolysis. The methanolysis of PET produces dimethyl terephthalate (DMT) and mono-ethylene glycol (MEG).

In an embodiment, the chemical depolymerization is carried out by hydrolysis. As an example, the chemical alkaline hydrolysis of PET, also called saponification, produces terephthalic acid salts and ethylene glycol (MEG). During the saponification reaction, the polyester is reacted with a strong base of alkali metals, such as NaOH or KOH, in the presence of water.

In a particular embodiment, the depolymerization step comprises contacting the at least one polyester having at least one unit of MEG with a chemical agent in an alkaline medium as starting reaction medium comprising an alkali such as KOH, NaOH, NH₄OH or LiOH.

In a particular embodiment, the depolymerizing agent is a chemical depolymerizing agent. Particularly, the chemical agent is a catalyst selected from metallic catalysts, stable and not toxic hydrosilanes (PMHS. TMDS), and other catalysts such as commercially available B(C₆F₅)₃ and [Ph₃C₊·B(C₆F₅)₄⁻] catalysts. Particularly, the catalyst is selected from alkoxide, carbonate, acetate, hydroxide, alkaline metal oxide, alkaline earth metal, calcium oxide, calcium hydroxide, calcium carbonate, sodium carbonate, iron oxide, zinc acetate, zeolite. In some embodiments, the catalyst used in the depolymerization process of the present invention comprises at least one of germanium compounds, titanium compounds, antimony compounds, zinc compounds, cadmium compounds, manganese compounds, magnesium compounds, cobalt compounds, silicon compounds, tin compounds, lead compounds, and aluminum compounds. Particularly, the catalyst comprises at least one of germanium dioxide, cobalt acetate, titanium tetrachloride, titanium phosphate, titanium tetrabutoxide, titanium tetraisopropoxide, titanium tetra-n-propoxide, titanium tetraethoxide, titanium tetramethoxide, a tetrakis(acetylacetonato)titanium complex, a tetrakis(2,4-hexanedionato)titanium complex, a tetrakis(3,5-heptanedionato)titanium complex, a dimethoxybis(acetylacetonato)titanium complex, a diethoxybis(acetylacetonato)titanium complex, a diisopropoxybis(acetylacetonato)titanium complex, a di-n-propoxybis(acetylacetonato)titanium complex, a dibutoxy bis(acetylacetonato)titanium complex, titanium dihydroxybisglycolate, titanium dihydroxybisglycolate, titanium dihydroxybislactate, titanium dihydroxybis(2-hydroxypropionate), titanium lactate, titanium octanediolate, titanium dimethoxy bistricthanol aminate, titanium diethoxybistriethanol aminate, titanium dibutoxybistriethanol aminate, hexamethyl dititanate, hexacthyl dititanate, hexapropyl dititanate, hexabutyl dititanate, hexaphenyl dititanate, octamethyl trititanate, octacthyl trititanate, octapropyl trititanate, octabutyl trititanate, octaphenyl trititanate, a hexaalkoxy dititanate, zinc acetate, manganese acetate, methyl silicate, zinc chloride, lead acetate, sodium carbonate, sodium bicarbonate, acetic acid, sodium sulphate, potassium sulphate, zeolites, lithium chloride, magnesium chloride, ferric chloride, zinc oxide, magnesium oxide, calcium oxide, barium oxide, antimony trioxide, and antimony triacetate. Alternatively, the catalyst is selected from nanoparticules. The chemical agent can be selected from any catalyst known by a person of the art for having the capacity to degrade and/or depolymerize the target polymer.

At the end of an alkaline chemical depolymerizing step, a “reaction solution” is obtained and comprises monomers of MEG and dicarboxylic acids salts such as terephthalic acid salts.

In another embodiment, the depolymerization of the polyester having at least one unit of MEG is a biological depolymerization, preferably a hydrolysis, more preferably an enzymatic depolymerization. Preferably, the biological depolymerization is an alkaline enzymatic depolymerization.

The term “biological depolymerization” refers to a process by which the depolymerization of at least one polyester having at least one unit of MEG is performed by contacting said at least one polyester having at least one unit of MEG with a biological agent capable of degrading said polyester. The biological depolymerization is carried out by hydrolysis. In a preferred embodiment, the hydrolysis is an alkaline hydrolysis. As an example, the biological alkaline hydrolysis of PET produces terephthalic acid (TA) salts and ethylene glycol (MEG).

In a particular embodiment, the depolymerizing agent is a biological depolymerizing agent. Particularly, the biological depolymerizing agent is an enzyme (i.e. a depolymerase). In this case, the biological depolymerization is called enzymatic depolymerization. Alternatively, or in addition, the biological depolymerizing agent is a microorganism that expresses and excretes said depolymerase. Preferably, the enzyme is able to degrade polyesters, more preferably the polyester object of the invention having at least one unit of MEG. As an example, the depolymerase, as an enzyme, is able to degrade PET, into monomeric forms i.e., TA, MEG, mono-2-hydroxyethyl terephthalate (MHET), and/or bis(2-hydroxyethyl) terephthalate (BHET).

In an embodiment, the enzyme is selected from esterases. In a preferred embodiment, the enzyme is selected from lipases or cutinases. In a particular embodiment, the plastic product is contacted with at least two different depolymerases.

In a particular embodiment, when the targeted polyester is PET, the depolymerase is an esterase. Particularly, the depolymerase is a cutinase, preferably a cutinase produced by a microorganism selected from Thermobifida cellulosityca, Thermobifida halotolerans, Thermobifida fusca, Thermobifida alba, Bacillus subtilis, Fusarium solani pisi, Humicola insolens, Sirococcus conigenus, Pseudomonas mendocina, Thielavia terrestris, Saccharomonospora viridis and Thermomonospora curvata, or any functional variant thereof. In another embodiment, the cutinase is selected from a metagenomic library such as LC-Cutinase described in Sulaiman et al., 2012 or the esterase described in EP3517608, or any functional variant thereof including depolymerases listed in WO 2018/011284, WO 2018/011281, WO 2020/021116, WO 2020/021117, WO 2020/021118, and WO2021005198. In another particular embodiment, the depolymerase is a lipase preferably produced by Ideonella sakaiensis or any functional variant thereof including depolymerases listed in WO2021005199. In another particular embodiment, the depolymerase is a cutinase produced by Humicola insolens, such as the one referenced A0A075B5G4 in Uniprot or any functional variant thereof. In another embodiment, the depolymerase is selected from commercial enzymes such as Novozym 51032 or any functional variant thereof. In an embodiment, the enzyme is selected from enzyme having a PET-degrading activity (PETase) and/or enzyme having a MHET-degrading activity (MHETasc).

In another embodiment, the depolymerizing agent is a microorganism that expresses and excretes the depolymerase. In the context of the invention the enzyme may be excreted in the culture medium or towards the cell membrane of the microorganism wherein said enzyme may be anchored. Said microorganism may naturally synthesize the depolymerase, or it may be a recombinant microorganism, wherein a recombinant nucleotide sequence encoding the depolymerase has been inserted, using for example a vector. For example, a nucleotide molecule, encoding the depolymerase of interest is inserted into a vector. e.g. plasmid, recombinant virus, phage, episome, artificial chromosome, and the like. Transformation of the host cell as well as culture conditions suitable for the host are well known to those skilled in the art. According to the invention, several microorganisms and/or several enzymes may be used together or sequentially to depolymerize different kinds of polymers contained in a same plastic article or in different plastic articles. In a particular embodiment, when the targeted polyester is PET, the depolymerization step is implemented at a temperature comprised between 20° C. and 90° C. preferably between 30° C. and 80° C. more preferably between 40° C. and 75° C. more preferably between 50° C. to 75° C. even more preferably between 60° C. to 75° C. Furthermore, the depolymerization step is preferably implemented at a pH between 5-11, preferably between 7-9, more preferably between 7-8.5, even more preferably between 7-8. In an embodiment, the depolymerization step is implemented at a pH between 6.5-9, preferably between 6.5-8.5, more preferably between 7-8, even more preferably between 7.5-8.5. Alternatively, the depolymerization step may be implemented under industrial and/or composting conditions.

In an embodiment, the depolymerization of the polyester having at least one unit of MEG is obtained from a biological depolymerization wherein the pH of the reaction medium is regulated between 6.5 and 9 by addition of a base in said reaction medium. Any base known by one skilled in the art may be used. Particularly, the base is selected from the group consisting of sodium hydroxide (NaOH), potassium hydroxide (KOH), lithium hydroxide (LiOH) or ammonia (NH₄OH). Advantageously, the base is sodium hydroxide (NaOH). The time required for the depolymerization step may vary depending on the polyester itself (i.e., origin, its composition, etc.), the type and amount of depolymerizing agent used, as well as various process parameters (i.e., temperature, pH, additional agents, etc.). One skilled in the art may easily adapt the depolymerization process step parameters. Examples of said process are described in WO 2014/079844, WO 2015/097104, WO 2015/173265, WO 2017/198786, WO 2020/094661, and WO 2020/094646

At the end of an alkaline biological depolymerizing step, a “reaction solution” is obtained and comprises monomers of MEG and/or dicarboxylic acids salts such as terephthalic acid salts and/or oligomers such as DEG or TEG and/or derivatives thereof including mono-2-hydroxyethyl terephthalate (MHET) and/or dimethyl terephthalate (DMT)) and/or colorants and/water. In a particular embodiment, the reaction solution of the depolymerization of at least one polyester having at least one unit of MEG comprises dicarboxylic acids salts and MEG and non-depolymerized polyester such as PET. In a preferred embodiment, the reaction solution of the depolymerization of at least PET comprises terephthalic acid salts, MEG and non-depolymerized PET.

In some embodiments, the reaction solution obtained at the end of the depolymerization step is used as solution obtained from the depolymerization of at least one polyester having at least one unit of MEG in a purifying process according to the invention. In alternative embodiments, the reaction solution obtained at the end of the depolymerization step is submitted to one or several preliminary steps to the purification process, as disclosed in the following section, to obtain the solution obtained from the depolymerization of at least one polyester having at least one unit of MEG to be used in a purifying process according to the invention.

### Optional Preliminary Steps to the Purification Process

The reaction solution obtained at the end of the depolymerization step can submitted to one or more steps before being submitted to the purifying process according to the invention. For example, said steps allow removing any solid residues, purifying the dicarboxylic acids salts and/or decoloring the reaction solution. Particular examples of said preliminary steps can be found in WO 2020/094661.

In an embodiment, the “reaction solution” can be submitted to one or more of the steps selected from the group consisting of filtration, purification of the filtrate, precipitation and evapo-concentration, prior to entering the MEG purification process of the invention. Preferably, the reaction solution can be submitted to one or more of the steps selected from the group consisting of filtration, decoloration, precipitation and evapo-concentration.

In an embodiment, the solution obtained from the depolymerization of at least one polyester having at least one unit of MEG is obtained from a reaction solution of a depolymerization of at least one polyester having at least one unit of MEG comprising dicarboxylic acids salts and MEG and submitted to one or more of the steps selected from the group consisting of filtration, decoloration, precipitation and evapo-concentration.

### —Filtration

By filtration it is meant to separate the solid non-depolymerized polyester, such as PET, from the liquid phase of the reaction medium (comprising dissolved dicarboxylic salts, such as TA salts, and MEG). The filtration threshold and the other conditions of the filtration, such as the optional presence of a filter aid, can be adapted by one skilled in the art. Alternatively, the separation of the non-depolymerized polyester can also be performed by centrifugation, or any other method known by one skilled in the art.

The filtrate which contains the dissolved dicarboxylic acid salts such as TA salts as well as the desired MEG is retained.

### —Purifying the Filtrate

The filtrate may be purified by subjecting it to one or several steps selected from ultrafiltration, decoloration, passage over ion exchangers and chromatography. Preferably, the filtrate is submitted to decoloration by adsorption on activated carbon. Any other equivalent method known by the skilled person can be used.

### —Precipitation of the Dicarboxylic Acids by Acidification of the Solution Followed by a Filtration

By precipitation by acidification, it is meant the precipitation of the dicarboxylic acids such as TA by addition of acids to the reaction solution to form a slurry. The acid can be selected from the group consisting of mineral acids, such as sulfuric acid, hydrochloric acid, phosphoric acid, or nitric acid, organic acids, such as acetic acid and mixtures thereof. Alternatively, the precipitation by acidification can be performed by CO₂ overpressure. This operation is followed by a filtration to remove the precipitated dicarboxylic acids. At the end, the filtrate contains the MEG with soluble residual salts (such as sodium sulphate) and MEG. Any other process to remove salts can be easily implemented by one skilled in the art.

For example, the filtrate containing the dicarboxylic acid salts, such as TA salts, can be submitted to all or some of the following steps:
1. Purification of the filtrate by submitting said solution to one or several steps selected from ultrafiltration, carbon adsorption, submission to an ion exchange resin and chromatography; and/or
2. Precipitation of the dicarboxylic acids, such as TA, contained in the purified filtrate by acidification with a mineral acid (which can be selected from the following: sulfuric acid, chloric acid, phosphoric acid or nitric acid) or with an organic acid (as acetic acid) or mixture thereof. The solution can also be acidified by CO₂ overpressure. This step also includes solubilization of the salts produced as the same time as dicarboxylic acid, such as TA, precipitation; and/or
3. Filtration of the solution comprising precipitated dicarboxylic acids, such as TA, to recover the dicarboxylic acids, such as TA, under solid form and the filtrate containing MEG with soluble residual salts (such as sodium sulphate). Accordingly, at least 80% of the dicarboxylic acids, such as TA, is removed from the reaction medium, preferably at least 90%, more preferably 95%, even more preferably greater than or equal to 98% prior to the process of the invention.

### —Evapo-Concentration Step to Obtain a Concentrated MEG Solution

The purpose of this step is to remove most of the dissolved salts, such as sodium sulphate, from the cited-above solution and to obtain a concentrated solution of MEG.

By evapo-concentration step, it is meant the operation of dehydration by heating the filtrate under vacuum. Once the desired pressure (vacuum) is reached, the water evaporates and the remaining salts, such as sodium sulphate, crystallize. The crystallized salts are then filtered out to obtain a concentrated solution of MEG (filtrate) and a cake of crystallized salts. This cake can be washed with water and the washing water is combined with the filtrate and then returned to the evaporator. The washing operation can be repeated several times by washing again the filtered cake, to combine it with the filtrate and to submit the mixture to an evapo-concentration step again. Advantageously, the washing operation is repeated three times to extract a maximum of the salts while minimizing the loss of MEG.

At the end, an aqueous concentrated solution of MEG is obtained.

For the following steps, it is referred to this aqueous concentrated solution of MEG as solution obtained from the depolymerization.

### Process for Recycling a Polymer-Containing Material Comprising at Least One Polyester Having at Least One Unit of MEG

It is also an object of the present invention to provide a process for recycling a polymer-containing material, such as a plastic product, comprising at least one polyester having at least one unit of MEG, the process comprises the following steps of:
a.1) Submitting the polyester to a depolymerization step to obtain a reaction solution comprising dicarboxylic acid salts, MEG and solid components,
b.1) Submitting the reaction solution obtained in step (a.1) to a filtration to remove solid components and obtain a filtrate,
c.1) Purifying the filtrate obtained in step (b.1), preferably through one or several steps selected from ultrafiltration, adsorption on activated carbon, submission to an ion exchange resin and chromatography, to obtain a purified filtrate,
d.1) Precipitating the dicarboxylic acid by acidification of the purified filtrate obtained in step (c.1), to obtain a slurry,
e.1) Submitting the slurry obtained in step (d.1) to a filtration to remove the precipitated dicarboxylic acid, to obtain a filtrate,
f.1) Submitting the filtrate obtained in step (e.1) to at least one evapo-concentration step to obtain an MEG concentrated solution,
g.1) Purifying the MEG of the concentrated solution obtained in step (f.1) by the process of the invention described above, to recover a purified MEG.

Particularly, the step (g.1) step comprises the following steps of:
a) Submitting the solution obtained in step (f.1) to at least one evapo-condensation step to obtain a bottom fraction and a condensed overhead fraction,
b) Contacting the condensed overhead fraction obtained in step (a) with a resin, to obtain a solution,
c) Submitting the solution obtained in step (b) to at least one distillation step to obtain a distillate, and
d) Recovering the purified MEG from the distillate obtained in step (c).

Steps d.1) and e.1) are of particular importance to obtain a MEG concentrated solution containing a low amount of salts, preferably substantially free of salts, such as TA salts produced from said depolymerization in order to obtain a MEG having the desired properties. Here, “substantially free” means that the MEG concentrated solution obtained at step f.1) comprises 3 wt. % or less of salts, such as 1 wt. % or less, such as 0.8 wt. % or less, such as 0.7 wt. % or less, such as 0.6 wt. % or less, such as 0.5% wt. % or less based on the total weight of the solution.

In an embodiment, the MEG concentrated solution obtained at step f.1) comprises 65 wt. % or more of MEG, such as 68 wt. % or more, such as 70 wt. % or more, such as 75 wt. % or more, such as 80 wt. % or more based on the total weight of the solution.

In an embodiment, the MEG concentrated solution obtained at step f.1) comprises 65 wt. % or more of MEG and DEG, such as 68 wt. % or more, such as 70 wt. % or more, such as 75 wt. % or more, such as 80 wt. % or more based on the total weight of the solution.

All the embodiments exposed above in connection with the process for purifying MEG from a solution obtained from the depolymerization of at least one polyester having at least one unit of MEG, also apply to the step (g.1) of the process for recycling a polymer-containing material comprising at least one polyester having at least one unit of MEG. In a preferred embodiment, the polyester having at least one unit of MEG is PET. In some embodiments, in step c), the solution obtained from step (b) is submitted to a double distillation, wherein the second one is a distillation with two sections.

The depolymerization step of step a.1) is performed such as described in the above section, i.e., chemical or biological depolymerization step. Preferably, the depolymerization step a.1) is performed in alkaline conditions by chemical or biological depolymerization.

In a preferred embodiment, the depolymerization step a.1) is a hydrolysis in alkaline conditions. In another preferred embodiment, it is a biological depolymerization step, i.e., an enzymatic depolymerization. Alternatively, the depolymerization step a.1) is a chemical hydrolysis in alkaline conditions such as saponification.

In an embodiment, the polymer-containing material comprising at least one polyester having at least one unit of MEG is a plastic product.

In a particular embodiment, when the polyester having at least one unit of MEG is a PET, the dicarboxylic acid is terephthalic acid (TA), the dicarboxylic acid salts are terephthalic acid salts. In such case, the solid components removed in step (b.1) comprise non-depolymerized PET.

According to the invention, the filtration of step b.1) and e.1), the purification of the filtrate of step c.1), the precipitating by acidification of step d.1) and/or the evapo-concentration of step f.1) are performed such as described in the above section, i.e, preliminary step to the purification process of the invention. In a preferred embodiment, the process for recycling a polymer-containing material comprising at least one polyester having at least one unit of MEG is performed in order from a.1) to g.1).

### Applications and Uses of the Purified MEG

According to the invention, the recovered MEG may be reused to synthesize polymers, particularly polyesters comprising at least one unit of MEG as described above. One skilled in the art may easily adapt the process parameters to the monomers/oligomers and the polymers to synthesize.

It is also an object of the invention to provide a polyester comprising at least one unit of MEG, preferably PET, polymerized from the MEG purified by the process of the invention.

In an embodiment, the recovered MEG may be reused as an antifreeze agent.

In an embodiment, the recovered MEG may be used as a desiccant in the gas industry.

The present invention also concerns the use of the purified MEG obtained by a process according to the invention to produce a polyester containing at least one unit of MEG.

### Polyester Comprising at Least One Unit of MEG

It is also an object of the present invention to provide a polyester comprising at least one unit of MEG, wherein the MEG is purified MEG obtained by a process according to the invention.

The following examples are intended to illustrate but not limit the disclosed embodiments.

### EXAMPLES

### Example A: Process for Recycling a Plastic Product Comprising a Polyester According to the Invention

### Experimental Part

### A—Preliminary Step: Preparation and Transformation of the PET Wastes

In example A1, ground PET trays comprising >98% of PET have been used. They were dry blended with 1% by weight of citric acid (Orgater exp 141/183 from Adeka) in powder form, based on the total weight of said composition, before being extruded using a twin-screw extruder Leistritz ZSE 18 MAXX which comprises nine successive heating zones (Z1-Z9) and a head (Z10) wherein the temperature may be independently controlled and regulated in each zone leading to an extruded foamed composition. The screw speed rate was set to 110 rpm, and total flow rate to 4 kg/h. The molten polymer arrived in the screw head (Z10) comprising a die plate with one hole of 3.5 mm and was immediately immersed in a 2 m long cold-water bath (10° C.). The resulting extrudate was granulated into 2-3 mm solid pellets.

Said pellets were then submitted to steps B to D.

In examples A2 and A3, washed and colored flakes from PET bottle waste comprising >98% of PET were micronized using an Ultra Centrifugal Mill ZM 200 system to a fine powder <500 μm size.

The powder was then submitted to steps B to D.

### B—Enzymatic Depolymerization Process of PET Wastes (Step a1)

The degrading process was carried out in a 1000 L reactor (800 L usable volume) using a variant of LC-Cutinase (Sulaiman et al., Appl Environ Microbiol. 2012 March). Such variant (herein after “LCC-ICCIG”) corresponds to the enzyme of SEQ ID N°1 with the following mutations F208I+D203C+S248C+V170I+Y92G and was expressed in Trichoderma reesei.

At the beginning of the degrading process, the transformed PET wastes were added at a concentration of 150 g/kg based on the total weight of reaction medium, and LCC-ICCIG was added at a concentration of 1 mg/g PET. During all the depolymerization step, agitation speed was regulated at 130 rpm, the temperature was regulated at 60° C. and the pH of the reaction medium was regulated at pH 8+0.05 by addition of NaOH solution at 25%.

The depolymerization rate was monitored by measuring the amount of NaOH added in the reaction medium to neutralize the terephthalic acid (TA) produced by the depolymerization.

The depolymerization rate after 48 h was 98%.

At the end of the depolymerization, the reaction solution comprises TA salts as dicarboxylic acid salts, MEG, and non-depolymerized PET in the form of solid component.

### C-Preliminary Steps to the MEG Purification Process: Removing Non Depolymerized PET and Terephthalic Acid (TA) and Sodium Sulphate Salts

### —Filtration (Step b1)

The reaction solution was then filtrated to separate the solid phase of the reaction solution in the reactor (mainly the residual non-depolymerized PET waste) from the liquid phase of the reaction solution (comprising solubilized TA salts and MEG).

The solid components removed from the fluid were eliminated. The filtrate which contained the solubilized TA salts as well as the desired MEG was retained.

### —Purifying the Filtrate by Adsorption on Activated Carbon (Step c1)

The filtrate containing the dissolved TA salts and MEG was purified by passing the filtrate through an activated carbon 1 L column, at room temperature.

### —Precipitation of the TA by Acidification of the Solution (Step d1) Followed by a Filtration (Step e1)

The purified filtrate was then acidified to precipitate the TA in the solution by adding sulphuric acid 98% until reaching a final pH between 2.7 and 3.

As a result, TA precipitate was formed and then filtered out from the slurry.

At the end, the filtrate contains the MEG with dissolved residual salts such as sodium sulphate.

### —Evapo-Concentration Step to Obtain a Concentrated MEG Solution (Step f1)

A first evaporation stage was carried out until crystals appeared by heating the product up to 80° C. while stirring and by progressively placing the evaporator under vacuum until pressure reaches approximately 300 mbar abs.

The obtained slurry was cooled overnight to crystallize the remaining dissolved sodium sulphate. The formed sodium sulphate crystals were filtered and then washed. The washing water and the filtrate containing the MEG were then returned to the evaporator for the next stage.

These operations (evaporating/filtering/washing the sodium sulphate crystals) were repeated three times to extract a maximum of the dissolved salts.

### D—Process for Purifying MEG (Step g1)

### Example A1—Process for Purifying the MEG Produced by Enzymatic Depolymerization of PET Trays

The “concentrated solution of MEG” obtained after the evapo-concentration step, as referred to in the previous section, will be called hereafter “the solution”.

The composition of the solution entering the process according to the invention is shown in Table 1.

TABLE 1
Composition of the concentrated solution of MEG
entering the process of purifying the MEG.

| Main components | Quantity (mass) |
|---|---|
| Water | 31.5% |
| MEG | 65.4% |
| DEG & Heavy components | 2.6% |
| Dry matter including Na₂SO₄ salts and heavy | 0.5% |
| impurities | |

Said solution was submitted to the process of the invention comprising the following steps:

### (a) Evapo-Condensation Step

The evaporation step was performed on a continuous glass made wiped film evaporator. The evaporator is heated with hot oil circulating in the double jacket. The product is fed continuously in the evaporator.

The evaporation of the solution was performed in two stages having different conditions such as described in Table 2. The overhead fraction resulting from the first evaporation stage was condensed to obtain a first condensate while the bottom fraction was submitted to the second evaporation stage. The overhead fraction obtained from the second evaporation step was also condensed to obtain the second condensate.

Finally, the first and second condensates were combined.

TABLE 2
Conditions of the two steps of evaporation

| Evaporation conditions: | 1^{st} evaporation stage | 2^{nd} evaporation stage |
|---|---|---|
| Process pressure (mbar abs) | 100 | 100 |
| Heating temperature (° C.) | 161-165 | 188-193 |

The heating temperature corresponds to the temperature of the hot oil circulating in the double jacket.

The condensations were performed using water-cooled condensing unit.

(b) Contacting with a Resin

The condensate obtained from step (a) was then passed through a 50 mm diameter/500 mm high glass column loaded with 375 g of PUROLITE A860 under its OH-form, at room temperature. The flow was set so that the velocity in the column is 1 m/h.

### (c) Distillations

A glass column distillation, i.e., Pro-pak distillation packing 0.16 inch with 1200 mm (3*400 mm) height, was used to perform two continuous distillation steps on the solution obtained from step (b).

The boiler of the column is heated with a hot oil circulated in the double jacket of the boiler.

The conditions of the two distillation steps are described in Table 3.

The column was fed at 1.3 kg/h.

TABLE 3
Conditions of the two distillation steps.

| | 1^{st} distillation step | 2^{nd} distillation step |
|---|---|---|
| Process Pressure (mbar abs) | 205 | 100 |
| Bottom temperature (° C.) | 153 | 136 |
| Heating temperature (° C.) | 175 | 200 |
| Reflux ratio | 1 | 1 |

The heating temperature corresponds to the temperature of the hot oil circulating in the double jacket.

### (d) Recovering the Purified MEG

The distillate comprising the purified MEG recovered from the process of the invention has the characteristics described in Table 4. The results are compared to the desired specification values of MEGs used to produce PET and plastic bottles. These specification values can be found in Material Data Sheet of commercial mono-ethylene glycol polyester grade.

TABLE 4
Analysis of the MEG purified by the process of the invention

| | Purified MEG | Petrochemical | | |
|---|---|---|---|---|
| | values of | sourced | | |
| Specifications | Example A1 | MEG Values | | Method |
| MEG Purity [wt-%] | >99.9% | 99.90 | min. | By difference: |
| | | | | according to |
| | | | | ASTM E-2409 |
| DEG [wt-%] | <0.05% | 0.05 | max. | ASTM E-2409 |
| Color, Pt—Co Units - Apha index | 1 | 5 | max. | ASTM D-5386 |
| UV transmission at 220 nm, % T | 76.8% | 70 | min. | ASTM E-2193 |
| UV transmission at 250 nm, % T | 95.9% | 90 | min. | ASTM E-2193 |
| UV transmission at 275 nm, % T | 95.9% | 94 | min. | ASTM E-2193 |

In conclusion, the process according to the invention leads to a purity greater than 99.9% which is like that of the petrochemical sourced MEG that is commercialized for producing PET and plastic bottles. In addition, the process of the invention allows to meet the other specified specification values as illustrated in table 4.

### Example A2—Process for Purifying the MEG Produced by Enzymatic Depolymerization of PET Bottles

For this example, the composition of the solution entering the purification section is shown in Table 5.

TABLE 5
Composition of the concentrated solution
of MEG entering the purification section.

| Main components | Quantity (mass) |
|---|---|
| Water | QSP |
| MEG | 80.8% |
| DEG & Heavy components | 3% |
| Dry matter (salts) | <1% |

Said solution was submitted to the process of the invention comprising the following steps:

### (a) Evapo-Condensation Step

The evaporation step was performed on a batch stainless steel evaporator. The 100 L evaporator is equipped with an agitation and heated with hot oil circulating in the double jacket.

The evaporation was performed as described in Example A1 with the conditions disclosed in Table 6 below.

TABLE 6
Conditions of the two evaporation steps.

| | 1^{st} evaporation stage | 2^{nd} evaporation stage |
|---|---|---|
| Process pressure (mbar abs) | 200 | 75 |
| Bottom temperature (° C.) | 164 | 136 |
| Heating temperature (° C.) | 170 | 170 |

The heating temperature corresponds to the temperature of the hot oil circulating in the double jacket.

The condensations were performed using water-cooled tube condenser.

(b) Contacting with a Resin

The deacidification step has been performed in an 8 L glass column loaded with 3.51 of PUROLITE A860 under its OH-form. The condensate obtained during step (a) was fed continuously in the column at room temperature at a flow set so that the velocity in the column was 0.8 m/h.

### (c) Distillations

A glass column distillation was used to perform two distillation operations. The column that was used to perform these experiments has the following characteristics:
Diameter: 50 mm
Packing: Sulzer packing BX
Useful packing height: 1000 mm per section

The boiler of the column is heated with a hot oil circulated in the shell of the falling film evaporator.

The conditions of the two distillation steps are described in Table 7.

TABLE 7
Conditions of the two distillation steps.

| | 1^{st} distillation step | 2^{nd} distillation step |
|---|---|---|
| Process Pressure (mbar abs) | 150 | 130 |
| Heating temperature (° C.) | 207-210 | 205 |
| Bottom temperature (° C.) | 143.5 | 140 |
| Reflux ratio | 0.2 | 0.07 |

The heating temperature corresponds to the temperature of the hot oil circulating in the double jacket.

(c′) Polishing Step

The distillate obtained from the previous step (c) was submitted to an additional polishing step. Said step has been performed in a 2.5 cm diameter glass column loaded with activated carbon. It was fed continuously in the column at room temperature. The flow was set at 0.6 l/h so that the velocity in the column was 1.2 m/h.

### (d) Recovering the Purified MEG

The purified MEG recovered from the process of the invention was analyzed and has the characteristics described in Table 8. As for example A1, the results are compared to the desired specification values of petrochemical sourced MEGs classically used to produce PET and plastic bottles.

TABLE 8
Analysis of the MEG purified by the process of the invention

| | Purified MEG | Petrochemical | |
|---|---|---|---|
| | values of | sourced MEG | |
| Specifications | Example A2 | values | |
| MEG Purity, wt-% | >99.9% | 99.90 | min. |
| DEG, wt % | Not detected | 0.05 | max. |
| Color, Pt—Co units - Apha index | 0 | 5 | max. |
| UV transmission at 220 nm, % T | 80% | 70 | min. |
| UV transmission at 250 nm, % T | >96% | 90 | min. |
| UV transmission at 275 nm, % T | >98% | 94 | min. |

In conclusion, the process for the purification of MEG according to the invention leads to a purity greater than 99.9% which is similar to the petrochemical sourced MEG that is classically used to produce PET and plastic bottles. In addition, the process of the invention allows to meet the other specified specification values as illustrated in Table 8.

Furthermore, clear bottles have been successfully produced using the MEG purified according to the invention and TA purified from a depolymerization process as described in WO 2014/079844, WO 2015/097104, WO 2015/173265, WO 2017/198786, WO 2020/094661, and WO 2020/094646.

### Example A3—Process for Purifying the MEG Produced by Enzymatic Depolymerization of PET Bottle Flakes Including a Distillation with Two Sections

The composition of the solution entering the process according to the invention is shown in Table 9.

TABLE 9
Composition of the concentrated solution of MEG
entering the process of purifying the MEG.

| Main components | Quantity (mass) |
|---|---|
| Water | QSP |
| MEG | 81% |
| DEG | About 4% |
| Heavy components | About 2% |
| Dry matter including Na₂SO₄ salts and heavy | <1% |
| impurities | |

Said solution was submitted to the process of the invention comprising the following steps:

### (a) Evapo-Condensation Step

The evaporation step was performed on a continuous 0.5 m² stainless steel made wiped film evaporator. The evaporator is heated with steam circulating in the double jacket. The product is fed continuously in the evaporator.

The evaporation of the solution was performed in two stages having different conditions such as described in Table 10. The overhead fraction resulting from the first evaporation stage was condensed to obtain a first condensate while the bottom fraction was submitted to the second evaporation stage. The overhead fraction obtained from the second evaporation step was also condensed to obtain the second condensate. Finally, the first and second condensates were combined.

TABLE 10
Conditions of the two steps of evaporation

| Evaporation conditions: | 1^{st} evaporation stage | 2^{nd} evaporation stage |
|---|---|---|
| Process pressure (mbar abs) | 100 | 65 |
| Heating temperature (° C.) | 161-165 | 150-160 |
| Feed flowrate (kg/hr) | 40 | 23 |

The heating temperature corresponds to the steam temperature circulating in the double jacket.

The condensations were performed using water-cooled condensing unit.

(b) Contacting with a Resin

The condensate obtained from step (a) was then passed through a high glass column loaded with PUROLITE A860 under its OH-form, at room temperature. The flow was set so that the velocity in the column is 1 m/h.

### (c) Distillations

Set-up used for the two consecutive distillations is based on example A2, introducing a supplementary upper section on the second distillation so that MEG is collected by a side take-off.

The boiler of the column is heated with hot oil or steam circulating in the double jacket.

The conditions of the two distillation steps are described in Table 11.

TABLE 11
Conditions of the two distillation steps.

| | 1^{st} distillation step | 2^{nd} distillation step |
|---|---|---|
| Process Pressure (mbar abs) | 205 | 100 |
| Bottom temperature (° C.) | 153 | 169 |
| Distillate temperature (° C.) | 60 | 132 |
| Reflux ratio | 0.2 | 0.5 |

### (d) Recovering the Purified MEG

The distillate comprising the purified MEG recovered from the process of the invention has the characteristics described in Table 12. The results are compared to the desired specification values of MEGs used to produce PET and plastic bottles. These specification values can be found in Material Data Sheet of commercial mono-ethylene glycol polyester grade.

TABLE 12
Analysis of the MEG purified by the process of the invention

| | Purified MEG | Petrochemical | | |
|---|---|---|---|---|
| | values of | sourced | | |
| Specifications | Example A3 | MEG Values | | Method |
| MEG Purity [wt. %] | >99.9% | 99.90 | min. | By difference: |
| | | | | according to |
| | | | | ASTM E-2409 |
| DEG [%] | <0.01% | 0.05 | max. | ASTM E-2409 |
| Color, Pt—Co Units - Apha index | 2 | 5 | max. | ASTM D-5386 |
| UV transmission at 220 nm, % T | 80% | 70 | min. | ASTM E-2193 |
| UV transmission at 250 nm, % T | 91% | 90 | min. | ASTM E-2193 |
| UV transmission at 275 nm, % T | 94% | 94 | min. | ASTM E-2193 |

Additionally, the recovered purified MEG exhibits an alpha-boiling value below 20. Such a specification can be easily measured by the skilled person in the art. For instance, the alpha boiling can be measured according to ASTM D-5386 method after heating the sample at 198° C. for 4 hours.

In conclusion, the process according to the invention leads to a purity greater than 99.9% which is like that of the petrochemical sourced MEG that is commercialized for producing PET and plastic bottles. In addition, the process of the invention allows to meet the other specified specification values as illustrated in Table 12.

1-15. (canceled)

16. A process for purifying mono-ethylene glycol (MEG) from a depolymerization solution of at least one polyester having at least one unit of MEG, the process comprises the following steps of:
(a) submitting said depolymerization solution to at least one evapo-condensation step to obtain a bottom fraction and a condensed overhead fraction,
(b) contacting the condensed overhead fraction obtained in step (a) with a resin, to obtain a solution,
(c) submitting the solution obtained in step (b) to at least one distillation step to obtain a distillate, and
(d) recovering the purified MEG from the distillate obtained in step (c).

17. The process according to claim 16, further comprises a step (c′) wherein the distillate obtained in step (c) is submitted to one or more steps selected from the group consisting of distillation, hydrogenation, dehydration and decoloration before recovery step (d), wherein said decoloration step is performed by activated carbon adsorption.

18. The process according to claim 16, wherein the polyester having at least one unit of MEG is selected from the group consisting of polyethylene terephthalate (PET), polyethylene adipate (PEA), polyethylene-2,5-furanoate (PEF), and polyethylene naphthalate (PEN).

19. The process according to claim 18, wherein the polyester having at least one unit of MEG is PET.

20. The process according to claim 16, wherein the depolymerization is a biological depolymerization, a hydrolysis, an enzymatic depolymerization, or an alkaline enzymatic depolymerization.

21. The process according to claim 16, wherein the depolymerization is a chemical depolymerization, a hydrolysis, an alkaline chemical hydrolysis, or saponification.

22. The process according to claim 16, wherein the depolymerization solution is a reaction solution of the depolymerization of at least one polyester having at least one unit of MEG comprising dicarboxylic acids salts and MEG and submitted to one or more of the steps selected from the group consisting of filtration, decoloration, precipitation and evapo-concentration.

23. The process according to claim 16, wherein the evaporation of the evapo-condensation of step (a) is performed in a thin-film evaporator, a batch evaporator, a forced circulation evaporator or a flash evaporator.

24. The process according to claim 16, wherein step (a) comprises one additional evapo-condensation step, wherein the bottom fraction obtained from the first evapo-condensation step is submitted to a second evapo-condensation step, and wherein the condensed overhead fractions obtained from the first and second evapo-condensation steps are mixed prior to being submitted to step (b).

25. The process according to claim 24, wherein the bottom fraction obtained from the first evapo-condensation step is filtrated prior to being submitted to the second evapo-condensation step and/or wherein the evaporation of the second evapo-condensation step is performed in a thin-film evaporator.

26. The process according to claim 16, wherein step (b) is performed by passing the condensed overhead fraction obtained in step (a) through an ion exchange resin or a strong anion exchange resin.

27. The process according to claim 16, wherein the evaporation of the first and/or of the second evapo-condensation steps of step (a) and/or the distillation step(s) of step (c) is/are performed under vacuum conditions.

28. The process according to claim 16, wherein said process comprises:
(a) submitting said depolymerization solution to at least one evapo-condensation step to obtain a bottom fraction and a mixture of condensed overhead fractions,
(b) contacting the condensed overhead fractions obtained in step (a) with a strong anion exchange resin to obtain a solution,
(c) submitting the solution obtained in step (b) to a double distillation, the second distillation being optionally a distillation with two sections, and
(d) recovering purified MEG from the distillate obtained in step (c).

29. A process for recycling a polymer-containing material comprising at least one polyester having at least one unit of MEG, the process comprising the following steps of:
(a.1) submitting the polyester having at least one unit of MEG to a depolymerization step to obtain a reaction solution comprising dicarboxylic acid salts, MEG and solid components,
(b.1) submitting the reaction solution obtained in step (a.1) to a filtration to remove solid components and obtain a filtrate,
(c.1) purifying the filtrate obtained in step (b.1) through one or several steps selected from ultrafiltration, adsorption on activated carbon, submission to an ion exchange resin and chromatography, to obtain a purified filtrate,
(d.1) precipitating the dicarboxylic acid by acidification of the purified filtrate obtained in step (c.1), to obtain a slurry,
(e.1) submitting the slurry obtained in step (d.1) to a filtration to remove the precipitated dicarboxylic acid, to obtain a filtrate,
(f.1) submitting the filtrate obtained in step (e.1) to at least one evapo-concentration step to obtain an MEG concentrated solution,
(g.1) purifying the MEG of the concentrated solution obtained in step (f.1) by the process according to claim 16 to recover a purified MEG.

30. The process according to claim 29, wherein the polyester having at least one unit of MEG is PET, the dicarboxylic acid is terephthalic acid (TA), and the dicarboxylic acid salts are terephthalic acid salts.

31. A method of producing a polyester comprising forming a polyester comprising purified MEG obtained by a process according to claim 16, said polyester containing at least one unit of MEG.


SEQUENCE LISTING (1 sequence)

SEQ ID NO: 1 - protein, 258 aa, synthetic construct
  REGION 1..258: note = LCCwt
SNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ